(19) United States
(12) Patent Application Publication
(10) Pub. No.: US 2024/0228404 A9
(43) Pub. Date: Jul. 11, 2024

(54) PROCESSES FOR PREPARING C2 TO C4 HYDROCARBONS AND PROCESS FOR PREPARING A FORMED HYBRID CATALYST

(71) Applicant: Dow Global Technologies LLC, Midland, MI (US)

(72) Inventors: Glenn Pollefeyt, Terneuzen (NL); Fang Du, Correggio (IT); Ewa Tocha, Terneuzen (NL); Alexey Kirilin, Terneuzen (NL); Christopher Ho, Midland, MI (US); David F. Yancey, Midland, MI (US); Davy L. S. Nieskens, Terneuzen (NL); Andrzej Malek, Midland, MI (US)

(73) Assignee: Dow Global Technologies LLC, Midland, MI (US)

(21) Appl. No.: 18/547,530

(22) PCT Filed: Feb. 18, 2022
(86) PCT No.: PCT/US2022/017011

§ 371 Date: Aug. 23, 2023

Related U.S. Application Data
(60) Provisional application No. 63/154,138, filed on Feb. 26, 2021.

(51) Int. Cl. C07C 1/12; B01J 21/04; B01J 21/06; B01J 23/10; B01J 29/85; B01J 35/00; B01J 35/40; B01J 37/00; B01J 37/02; B01J 37/08

(52) U.S. Cl. CPC C07C 1/12; B01J 21/04; B01J 21/066; B01J 23/10; B01J 29/85; B01J 35/19; B01J 35/40; B01J 37/0009; B01J 37/0201; B01J 37/088; B01J 2229/42; C07C 2523/08

(57) ABSTRACT

A process for preparing C₂ to C₄ hydrocarbons includes introducing a feed stream including hydrogen gas and a carbon-containing gas selected from the group consisting of carbon monoxide, carbon dioxide, and mixtures thereof into a reaction zone of a reactor, and converting the feed stream into a product stream including C₂ to C₄ hydrocarbons in the reaction zone in the presence of a formed hybrid catalyst. The formed hybrid catalyst includes a metal oxide catalyst component including gallium oxide and zirconia, a microporous catalyst component that is a molecular sieve having 8-MR (Membered Ring) pore openings, and a binder including alumina, zirconia, or both.

### CROSS-REFERENCE TO RELATED APPLICATIONS

This application claims priority to U.S. Provisional Patent Application No. 63/154,138, filed Feb. 26, 2021, the entire disclosure of which is hereby incorporated by reference.

### BACKGROUND

### Field

The present disclosure relates to processes that efficiently convert various carbon-containing streams to C₂ to C₄ hydrocarbons. In particular, the present disclosure relates to preparation of formed hybrid catalysts and application of process methods to achieve a high conversion of synthesis gas feeds resulting in good conversion of carbon and high yield of desired products.

### Technical Background

For a number of industrial applications, hydrocarbons are used, or are starting materials used, to produce plastics, fuels, and various downstream chemicals. Such hydrocarbons include C₂ to C₄ materials, such as ethylene, propylene, and butylenes (also commonly referred to as ethene, propene and butenes, respectively). A variety of processes for producing these lower hydrocarbons have been developed, including petroleum cracking and various synthetic processes.

Synthetic processes for converting feed carbon to desired products, such as hydrocarbons, are known. Different types of catalysts have been explored, as well as different kinds of feed streams and proportions of feed stream components.

However, these catalysts in themselves need to be loaded in reactors in a shaped form to reduce the pressure drop over the reactors. Further, many of these synthetic processes have low carbon conversion and much of the feed carbon either (1) does not get converted and exits the process in the same form as the feed carbon; (2) is converted to CO₂; or (3) these synthetic processes have low stability over time and the catalyst rapidly loses its activity for carbon conversion to desirable products. For example, many synthetic processes tend to have increased methane production—and, thus, decreased C₂ to C₄ hydrocarbon production—over time.

Accordingly, a need exists for processes and catalytic systems that include a single catalyst body having both microporous catalyst component and metal oxide catalyst component, rather than each components individually being formed, and have a high conversion of feed carbon to desired products, such as, for example, C₂ to C₄ hydrocarbons in combination with a high on stream stability of the catalyst.

### SUMMARY

Embodiments of the present disclosure address these and other needs by preparation of formed hybrid catalysts and processes using such a catalyst. A formed hybrid catalyst includes a combination of a metal oxide component, a microporous catalyst component, and a binder. The metal oxide component and the microporous catalyst component are combined into a single catalyst body using the binder. This formed hybrid catalyst can then be used for the direct conversion of a feed stream comprising hydrogen gas and a carbon-containing gas, such as syngas, to C₂ to C₄ hydrocarbons. The metal oxide component and the microporous catalyst component operate in tandem so that the formed hybrid catalyst is able to directly and selectively convert a feed stream comprising hydrogen and carbon-containing gas, such as syngas to C₂ to C₄ hydrocarbons with high olefin/paraffin ratio.

According to one or more aspects of the present disclosure, a process for preparing C₂ to C₄ hydrocarbons includes introducing a feed stream including hydrogen gas and a carbon-containing gas selected from the group consisting of carbon monoxide, carbon dioxide, and mixtures thereof into a reaction zone of a reactor, and converting the feed stream into a product stream including C₂ to C₄ hydrocarbons in the reaction zone in the presence of a formed hybrid catalyst. The formed hybrid catalyst includes a metal oxide catalyst component including gallium oxide and zirconia, a microporous catalyst component that is a molecular sieve having 8-MR (Membered Ring) pore openings, and a binder including alumina, zirconia, or both.

According to one or more other aspects of the present disclosure, a process for preparing a formed hybrid catalyst includes mixing a metal oxide catalyst component and a microporous catalyst component, wherein the metal oxide catalyst component includes gallium oxide and zirconia, and the microporous catalyst component includes a molecular sieve having 8-MR pore openings, adding a binder to the mixture of the metal oxide catalyst component and the microporous catalyst component to form a paste, wherein the binder is a colloidal solution, suspension, or gel of a binder precursor comprising oxides or hydroxides of aluminum, oxides or hydroxides of zirconium, or mixtures thereof, and extruding the paste to produce the formed hybrid catalyst after drying and subsequent calcination.

Additional features and advantages will be set forth in the detailed description which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### DETAILED DESCRIPTION

Reference will now be made in detail to embodiments of methods for preparing the formed hybrid catalyst and to processes for forming C₂ to C₄ hydrocarbons from a feed stream comprising hydrogen gas and a carbon-containing gas. As used herein, “a carbon-containing gas” refers to a gas selected from carbon monoxide, carbon dioxide, and mixtures thereof. In one embodiment, a process for preparing C₂ to C₄ hydrocarbons includes introducing a feed stream including hydrogen gas and a carbon-containing gas selected from the group consisting of carbon monoxide, carbon dioxide, and mixtures thereof into a reaction zone of a reactor, and converting the feed stream into a product stream including C₂ to C₄ hydrocarbons in the reaction zone in the presence of a formed hybrid catalyst. The formed hybrid catalyst includes a metal oxide catalyst component including gallium oxide and zirconia, a microporous catalyst component that is a molecular sieve having 8-MR pore openings, and a binder including alumina, zirconia, or both.

As used herein, “gallium oxide” refers to gallium in various oxidation states. In one embodiments, gallium oxide may include Ga₂O₃. In other embodiments, gallium oxide may include gallium in more than one oxidation state. For example, individual gallium may be in different oxidation states. Gallium oxide is not limited to comprising gallium in homogenous oxidation states.

The use of formed hybrid catalysts is known in the field of hydrocarbon products, such as diesel, or aromatics. However, many known formed hybrid catalysts are inefficient for forming C₂ to C₄ hydrocarbons, and particularly C₂ to C₄ olefins, from a feed stream comprising hydrogen gas and a carbon-containing gas, because they exhibit a low feed carbon conversion and/or deactivate quickly as they are used, such as, for example, by having an increase in methane production, which leads to a low olefin yield and low stability for a given set of operating conditions over a given amount of time. In contrast, formed hybrid catalysts disclosed and described herein exhibit a high and steady yield of particularly C₂ to C₄ olefins even as the catalyst time on stream increases when compared to hybrid catalysts where the metal oxide catalyst component and the microporous catalyst component are physically mixed (e.g., are not formed together into a formed hybrid catalyst). The preparation and composition of such formed hybrid catalysts used in embodiments is discussed below.

As a summary, formed hybrid catalysts closely couple independent reactions on each of the two independent catalysts. In the first step, a feed stream comprising hydrogen gas (H₂) and a carbon-containing bas selected from the group consisting of carbon monoxide (CO), carbon dioxide (CO₂), or a mixture of CO and CO₂, such as, for example, syngas, is converted into an intermediate(s) such as oxygenated hydrocarbons. In the subsequent step, these intermediates are converted into a product stream comprising hydrocarbons (mostly short chain hydrocarbons, such as, for example C₂ to C₄ hydrocarbons). The continued formation and consumption of the intermediate oxygenates formed in the first step by the reactions of the second step ensures that there is no thermodynamic limit on conversions.

In embodiments, the formed hybrid catalyst has a particle size from 0.5 millimeter (mm) to 6.0 mm, such as from 0.5 mm to 5.5 mm, from 0.5 mm to 5.0 mm, from 0.5 mm to 4.5 mm, from 0.5 mm to 4.0 mm, from 0.5 mm to 3.5 mm, from 0.5 mm to 3.0 mm, from 0.5 mm to 2.5 mm, from 0.5 mm to 2.0 mm, or from 0.5 mm to 1.5 mm. In embodiments, the formed hybrid catalyst has a particle size from 1.0 mm to 6.0 mm, such as from 1.0 mm to 5.5 mm, from 1.0 mm to 5.0 mm, from 1.0 mm to 4.5 mm, from 1.0 mm to 4.0 mm, from 1.0 mm to 3.5 mm, from 1.0 mm to 3.0 mm, from 1.0 mm to 2.5 mm, from 1.0 mm to 2.0 mm, or from 1.0 mm to 1.5 mm. In embodiments, the formed hybrid catalyst has a particle size from 1.5 mm to 3.0 mm, such as from 1.8 mm to 3.0 mm, from 2.0 mm to 3.0 mm, from 2.2 mm to 3.0 mm, from 2.5 mm to 3.0 mm, from 2.8 mm to 3.0 mm, from 1.5 mm to 2.8 mm, from 1.8 mm to 2.8 mm, from 2.0 mm to 2.8 mm, from 2.2 mm to 2.8 mm, from 2.5 mm to 2.8 mm, from 1.5 mm to 2.5 mm, from 1.8 mm to 2.5 mm, from 2.0 mm to 2.5 mm, from 2.2 mm to 2.5 mm, from 1.5 mm to 2.2 mm, from 1.8 mm to 2.2 mm, from 2.0 mm to 2.2 mm, from 1.5 mm to 2.0 mm, from 1.8 mm to 2.0 mm, or from 1.5 mm to 1.8. The particle size may be essentially the shortest dimension of the catalyst particle. For example, when the formed hybrid catalyst has a hollow cylinder or a ring shape, the particle size is the thickness of the hollow cylinder wall. When the formed hybrid catalyst has a spherical shape, the particle size is the diameter of the sphere. The particle size of the formed hybrid catalyst may be controlled by choice of the extrusion die diameter and measured by dynamic image analysis methods.

Formed hybrid catalysts include a metal oxide catalyst component, which converts the feed stream to oxygenated hydrocarbons, and a microporous catalyst component, which converts the oxygenated hydrocarbons to hydrocarbons. The metal oxide catalyst component is combined with a microporous catalyst component. There is accordingly a need for a metal oxide catalyst component that results in a high initial yield as well as a high stability when combined with a microporous catalyst component in a formed hybrid catalyst process. It should be understood that, as used herein, the “metal oxide catalyst component” includes metals in various oxidation states. In some embodiments, the metal oxide catalyst component may include more than one metal oxide and individual metal oxides within the metal oxide catalyst component may have different oxidation states. Thus, the metal oxide catalyst component is not limited to comprising metal oxides with homogenous oxidation states.

In embodiments, the metal oxide catalyst component has a particle size of less than 150 μm, less than 120 μm, or less than 100 μm. In some embodiments, the metal oxide catalyst component has a particle size of from 0.1 μm to 150 μm, from 0.1 μm to 120 μm, from 0.1 μm to 100 μm, from 1 μm to 150 μm, from 1 μm to 120 μm, from 1 μm to 100 μm, from 1 μm to 50 μm, from 5 μm to 150 μm, from 5 μm to 120 μm, from 5 μm to 100 μm, from 5 μm to 50 μm, from 10 μm to 150 μm, from 10 μm to 120 μm, from 10 μm to 100 μm, or from 10 μm to 50 μm. The particle size may refer to a maximum particle size. The particle size of the metal oxide catalyst component may refer to a physical dimension of metal oxide catalyst component, not to a crystal size of the metal oxide catalyst component. The particle size of the metal oxide catalyst component may be measured by laser diffraction or by passing the materials through an analytical sieve.

In embodiments, the metal oxide catalyst component has a particle size D₅₀, which is the median diameter or the medium value of the volumetric particle size distribution, of from 1 μm to 100 μm, from 1 μm to 90 μm, from 1 μm to 80 μm, or from 1 μm to 50 μm.

The metal oxide catalyst component comprises gallium oxide and zirconia (ZrO₂). As used herein, the zirconia used in embodiments disclosed and described herein in the metal oxide catalyst component of the formed hybrid catalyst is “phase pure zirconia”, which is defined herein as zirconia to which no other materials have intentionally been added during formation. Thus, “phase pure zirconia” includes zirconia with small amounts of components other than zirconium (including oxides other than zirconia) that are unintentionally present in the zirconia as a natural part of the zirconia formation process, such as, for example, hafnium (Hf). Accordingly, as used herein “zirconia” and “phase pure zirconia” are used interchangeably unless specifically indicated otherwise.

Without being bound by any particular theory, it is believed that the high surface area of zirconia allows the gallium oxide catalyst acting as part of formed hybrid catalyst to convert carbon-containing components to C₂ to C₄ hydrocarbons. It is believed that the gallium oxide and the zirconia help to activate one another, which results in improved yield for C₂ to C₄ hydrocarbons.

In embodiments disclosed herein, the composition of the metal oxide catalyst component is designated by a weight percentage of the gallium oxide metal to the pure zirconia (accounting for ZrO₂ stoichiometry). In one or more embodiments, the composition of the metal oxide catalyst component is designated by weight of gallium oxide per 100 grams (g) of zirconia. According to embodiments, the metal oxide catalyst component includes from 0.1 g gallium oxide to 30.0 g gallium oxide per 100 g of zirconia, such as 5.0 g gallium oxide to 30.0 g gallium oxide per 100 g of zirconia, 10.0 g gallium oxide to 30.0 g gallium oxide per 100 g of zirconia, 15.0 g gallium oxide to 30.0 g gallium oxide per 100 g of zirconia, 20.0 g gallium oxide to 30.0 g gallium oxide per 100 g of zirconia, or 25.0 g gallium oxide to 30.0 g gallium oxide per 100 g of zirconia. In some embodiments, the metal oxide catalyst component includes from 0.1 g gallium oxide to 25.0 g gallium oxide per 100 g of zirconia, such as from 0.1 g gallium oxide to 20.0 g gallium oxide per 100 g of zirconia, from 0.1 g gallium oxide to 15.0 g gallium oxide per 100 g of zirconia, from 0.1 g gallium oxide to 10.0 g gallium oxide per 100 g of zirconia, or from 0.1 g gallium oxide to 5.0 g gallium oxide per 100 g of zirconia. In some embodiments, the metal oxide catalyst component includes from 5.0 g gallium oxide to 25.0 g gallium oxide per 100 g of zirconia, such as from 10.0 g gallium oxide to 20.0 g gallium oxide per 100 g of zirconia. In some embodiments, the metal oxide catalyst component includes from 0.1 g gallium oxide per 100 g of zirconia to 5.00 g gallium oxide to 100 g zirconia, such as from 0.50 g gallium oxide per 100 g of zirconia to 5.00 g gallium oxide to 100 g zirconia, from 1.00 g gallium oxide per 100 g of zirconia to 5.00 g gallium oxide to 100 g zirconia, from 1.50 g gallium oxide per 100 g of zirconia to 5.00 g gallium oxide to 100 g zirconia, from 2.00 g gallium oxide per 100 g of zirconia to 5.00 g gallium oxide to 100 g zirconia, from 2.50 g gallium oxide per 100 g of zirconia to 5.00 g gallium oxide to 100 g zirconia, from 3.00 g gallium oxide per 100 g of zirconia to 5.00 g gallium oxide to 100 g zirconia, from 3.50 g gallium oxide per 100 g of zirconia to 5.00 g gallium oxide to 100 g zirconia, from 4.00 g gallium oxide per 100 g of zirconia to 5.00 g gallium oxide to 100 g zirconia, or from 4.50 g gallium oxide per 100 g of zirconia to 5.00 g gallium oxide to 100 g zirconia.

In embodiments disclosed herein, the composition of the metal oxide catalyst component is designated by a weight percentage of the lanthanum oxide metal to the pure zirconia (accounting for ZrO₂ stoichiometry). In one or more embodiments, the composition of the metal oxide catalyst component is designated by weight of lanthanum oxide per 100 grams (g) of zirconia. According to embodiments, the metal oxide catalyst component includes from 0.1 g lanthanum oxide to 10.0 g lanthanum oxide per 100 g of zirconia, such as 5.0 g lanthanum oxide to 10.0 g lanthanum oxide per 100 g of zirconia, 10.0 g lanthanum oxide to 30.0 g lanthanum oxide per 100 g of zirconia, 15.0 g lanthanum oxide to 30.0 g lanthanum oxide per 100 g of zirconia, 20.0 g lanthanum oxide to 30.0 g lanthanum oxide per 100 g of zirconia, or 25.0 g lanthanum oxide to 30.0 g lanthanum oxide per 100 g of zirconia. In some embodiments, the metal oxide catalyst component includes from 0.1 g lanthanum oxide to 25.0 g lanthanum oxide per 100 g of zirconia, such as from 0.1 g lanthanum oxide to 20.0 g lanthanum oxide per 100 g of zirconia, from 0.1 g lanthanum oxide to 15.0 g lanthanum oxide per 100 g of zirconia, from 0.1 g lanthanum oxide to 10.0 g lanthanum oxide per 100 g of zirconia, or from 0.1 g lanthanum oxide to 5.0 g lanthanum oxide per 100 g of zirconia. In some embodiments, the metal oxide catalyst component includes from 5.0 g lanthanum oxide to 25.0 g lanthanum oxide per 100 g of zirconia, such as from 10.0 g lanthanum oxide to 20.0 g lanthanum oxide per 100 g of zirconia. In some embodiments, the metal oxide catalyst component includes from 0.1 g lanthanum oxide per 100 g of zirconia to 5.00 g lanthanum oxide to 100 g zirconia, such as from 0.50 g lanthanum oxide per 100 g of zirconia to 5.00 g lanthanum oxide to 100 g zirconia, from 1.00 g lanthanum oxide per 100 g of zirconia to 5.00 g lanthanum oxide to 100 g zirconia, from 1.50 g lanthanum oxide per 100 g of zirconia to 5.00 g lanthanum oxide to 100 g zirconia, from 2.00 g lanthanum oxide per 100 g of zirconia to 5.00 g lanthanum oxide to 100 g zirconia, from 2.50 g lanthanum oxide per 100 g of zirconia to 5.00 g lanthanum oxide to 100 g zirconia, from 3.00 g lanthanum oxide per 100 g of zirconia to 5.00 g lanthanum oxide to 100 g zirconia, from 3.50 g lanthanum oxide per 100 g of zirconia to 5.00 g lanthanum oxide to 100 g zirconia, from 4.00 g lanthanum oxide per 100 g of zirconia to 5.00 g lanthanum oxide to 100 g zirconia, or from 4.50 g lanthanum oxide per 100 g of zirconia to 5.00 g lanthanum oxide to 100 g zirconia.

In view of the above, one method for making the gallium oxide and zirconia metal oxide component of the formed hybrid catalyst is by incipient wetness impregnation. In such a method, an aqueous mixture of a gallium precursor material, which, in embodiments, may be gallium nitrate (Ga(NO₃)₃) is added to zirconia powder in a dosed amount (such as dropwise) while stirring and mixing the zirconia particles. In other embodiments, the gallium oxide may be deposited or distributed on the zirconia oxide by chemical vapor deposition (CVD) method. However, the method for making the gallium oxide and zirconia metal oxide component of the formed hybrid catalyst is not particularly limited and any method that can apply a fine layer of gallium oxide on the surface of zirconium oxide can be used according to embodiments. It should be understood that the total amount of gallium precursor that is mixed with the zirconia particles will be determined on the desired target amount of gallium in metal oxide catalyst component.

As discussed previously, according to some embodiments, the zirconia particles include zirconia particles having a crystalline structure. In embodiments, the zirconia particles include zirconia particles having a monoclinic structure. In one or more embodiments, the zirconia particles consist essentially of or consist of crystalline zirconia particles, and in some embodiments, the zirconia particles consist essentially of or consist of monoclinic zirconia particles. According to some embodiments, the zirconia particles have a BET surface area that is greater than or equal to 5 meters squared per gram (m²/g), such as greater than 10 m²/g, greater than 20 m²/g, greater than 30 m²/g, greater than 40 m²/g, greater than 50 m²/g, greater than 60 m²/g, greater than 70 m²/g, greater than 80 m²/g, greater than 90 m²/g, greater than 100 m²/g, greater than 110 m²/g, greater than 120 m²/g, greater than 130 m²/g, or greater than 140 m²/g. According to some embodiments, the maximum BET surface area of the zirconia particles is 150 m²/g. Accordingly, in some embodiments, the BET surface area of the zirconia particles is from 5 m²/g to 150 m²/g, from 10 m²/g to 150 m²/g, from 20 m²/g to 150 m²/g, such as from 30 m²/g to 150 m²/g, from 40 m²/g to 150 m²/g, from 50 m²/g to 150 m²/g, from 60 m²/g to 150 m²/g, from 70 m²/g to 150 m²/g, from 80 m²/g to 150 m²/g, from 90 m²/g to 150 m²/g, from 100 m²/g to 150 m²/g, from 110 m²/g to 150 m²/g, from 120 m²/g to 150 m²/g, from 130 m²/g to 150 m²/g, or from 140 m²/g to 150 m²/g. In some embodiments, the BET surface area of the zirconia particles is from 5 m²/g to 140 m²/g, such as from 5 m²/g to 130 m²/g, from 5 m²/g to 120 m²/g, from 5 m²/g to 110 m²/g, from 5 m²/g to 100 m²/g, from 5 m²/g to 90 m²/g, from 5 m²/g to 80 m²/g, from 5 m²/g to 70 m²/g, from 5 m²/g to 60 m²/g, from 5 m²/g to 50 m²/g, from 5 m²/g to 40 m²/g, from 5 m²/g to 30 m²/g, from 5 m²/g to 20 m²/g, or from 5 m²/g to 10 m²/g. In some embodiments, the BET surface area of the zirconia particles is from 10 m²/g to 140 m²/g, from 20 m²/g to 130 m²/g, from 30 m²/g to 120 m²/g, from 40 m²/g to 110 m²/g, from 50 m²/g to 100 m²/g, from 60 m²/g to 90 m²/g, or from 70 m²/g to 80 m²/g.

Once the gallium precursor and zirconia particles are adequately mixed, the metal oxide catalyst component may be dried at temperatures less than 200 degrees Celsius (° C.), such as less than 175° C., less than 150° C., less than 100° C., or about 85° C. Subsequent to the drying, the metal oxide catalyst component is calcined at temperatures from 400° C. to 800° C., such as from 425° C. to 775° C., from 450° C. to 750° C., from 475° C. to 725° C., from 500° C. to 700° C., from 525° C. to 675° C., from 550° C. to 650° C., from 575° C. to 625° C., about 550° C., or about 600° C. After calcining, the composition of the mixed metal oxide catalyst component is determined and reported as a weight of gallium oxide taken as Ga₂O₃ referenced per 100 g of phase pure zirconia (simplified to the stoichiometry of ZrO₂) as previously disclosed above.

In embodiments, the metal oxide catalyst component may be made by mixing powders or slurries of a gallium precursor (such as gallium nitrate or gallium oxide) and zirconia. According to some embodiments, the zirconia particles include zirconia particles having a crystalline structure. In embodiments, the zirconia particles include zirconia particles having a monoclinic structure. In one or more embodiments, the zirconia particles consist essentially of or consist of crystalline zirconia particles, and in some embodiments, the zirconia particles consist essentially of or consist of monoclinic zirconia particles. The zirconia particles may, in embodiments, have the BET surface areas disclosed above. The powders or slurries may be vigorously mixed at high temperatures such from room temperature (approximately 23° C.) to 100° C. After the powders or slurries have been adequately mixed, the metal oxide catalyst component may be dried and calcined at temperatures from 400° C. to 800° C., such as from 425° C. to 775° C., from 450° C. to 750° C., from 475° C. to 725° C., from 500° C. to 700° C., from 525° C. to 675° C., from 550° C. to 650° C., from 575° C. to 625° C., or about 600° C. After calcining, the composition of the mixed metal oxide catalyst component is determined and reported as a weight of gallium oxide taken as Ga₂O₃ in reference to 100 g of phase pure zirconia (simplified to the stoichiometry of ZrO₂) as disclosed above.

It should be understood that according to embodiments, the metal oxide catalyst component may be made by other methods that eventually lead to intimate contact between the gallium precursor and zirconia. Some non-limiting instances include vapor phase deposition of Ga-containing precursors (either organic or inorganic in nature), followed by their controlled decomposition. Similarly, processes for dispersing liquid gallium metal can be amended by those skilled in the art to lead to intimate contact between the gallium precursor and zirconia.

Elements other than gallium oxide and zirconia may, in some embodiments, be present in the metal oxide catalyst component containing phase pure zirconia and gallium oxide. Such elements may be introduced to the phase pure zirconia before, during or after introducing gallium precursor to the composition. Sometimes such elements are added to direct and stabilize the crystallization of zirconia phase (e.g., Y-stabilized tetragonal ZrO₂).

In embodiments, the metal oxide catalyst component includes lanthanum. In other instances, additional elements from the group of rare earth, alkaline, and/or transition metals are co-deposited with gallium precursor or introduced only when the mixed composition including gallium oxide and zirconia has been prepared in the first place.

In one or more embodiments, after the metal oxide catalyst component has been prepared—such as, for example, by the methods disclosed above—the metal oxide catalyst component is mixed with a microporous catalyst component, and a binder to form a single catalyst. The microporous catalyst component is, in embodiments, selected from molecular sieves having 8-MR pore openings and having a framework type selected from the group consisting of the following framework types CHA, AEI, AFX, ERI, LEV, LTA, UFI, RTH, EDI, GIS, MER, RHO, and combinations thereof, the framework types corresponding to the naming convention of the International Zeolite Association. It should be understood that in embodiments, both aluminosilicate and silicoaluminophosphate frameworks may be used. Some embodiments may include tetrahedral aluminosilicates, ALPOs (such as, for example, tetrahedral aluminophosphates), SAPOs (such as, for example, tetrahedral silicoaluminophosphates), and silica-only based tectosilicates. In certain embodiments, the microporous catalyst component may be silicoaluminophosphate having a Chabazite (CHA) framework type. Examples of these may include, but are not necessarily limited to: CHA embodiments selected from SAPO-34 and SSZ-13; and AEI embodiments such as SAPO-18. Combinations of microporous catalyst components having any of the above framework types may also be employed. It should be understood that the microporous catalyst component may have different membered ring pore opening depending on the desired product. For instance, microporous catalyst component having 8-MR to 12-MR pore openings could be used depending on the desired product. However, to produce C₂ to C₄ hydrocarbons, a microporous catalyst component having 8-MR pore openings is used in embodiments.

The metal oxide catalyst component and the microporous catalyst component of the formed hybrid catalyst may be mixed together by any suitable means to achieve homogenous mixing of all the components prior to extrusion. The metal oxide catalyst component and the microporous catalyst component can be initially mixed as powders to achieve homogeneity in suitable dry mixer, such as a ribbon or plow mixer. The peptized binder precursor can be added to the mixture of the metal oxide catalyst component and the microporous catalyst component and mixed in a suitable heavy duty industrial mixer capable of handling thick paste formulations. Alternatively, the dry pre-mixed metal oxide catalyst component and the microporous catalyst component can be fed directly into the feeding screws of a screw extruder along with the peptized binder precursor composition and mixed directly in the screw extruder. The formed hybrid catalyst may be extruded into a desired shape by any suitable extrusion method. Examples of shapes include pellets, spherical, or near-spherical. In embodiments, the metal oxide catalyst component may include from 1.0 weight percent (wt %) to 80.0 wt % of the formed hybrid catalyst, such as from 5.0 wt % to 80.0 wt %, from 10.0 wt % to 80.0 wt %, from 15.0 wt % to 80.0 wt %, from 20.0 wt % to 80.0 wt %, from 25.0 wt % to 80.0 wt %, from 30.0 wt % to 80.0 wt %, from 35.0 wt % to 80.0 wt %, from 40.0 wt % to 80.0 wt %, from 45.0 wt % to 80.0 wt %, from 50.0 wt % to 80.0 wt %, from 55.0 wt % to 80.0 wt %, from 60.0 wt % to 80.0 wt %, from 65.0 wt % to 80.0 wt %, from 70.0 wt % to 80.0 wt %, or from 75.0 wt % to 80.0 wt %. In some embodiments, the metal oxide catalyst component includes from 1.0 wt % to 80.0 wt %, from 1.0 wt % to 75.0 wt %, from 1.0 wt % to 70.0 wt %, from 1.0 wt % to 65.0 wt %, from 1.0 wt % to 60.0 wt %, from 1.0 wt % to 55.0 wt %, from 1.0 wt % to 50.0 wt %, from 1.0 wt % to 45.0 wt %, from 1.0 wt % to 40.0 wt %, from 1.0 wt % to 35.0 wt %, from 1.0 wt % to 30.0 wt %, from 1.0 wt % to 25.0 wt %, from 1.0 wt % to 20.0 wt %, from 1.0 wt % to 15.0 wt %, from 1.0 wt % to 10.0 wt %, or from 1.0 wt % to 5.0 wt %. In some embodiments, the metal oxide catalyst component includes from 5.0 wt % to 80.0 wt % of the formed hybrid catalyst, such as from 10.0 wt % to 80.0 wt %, from 15.0 wt % to 80.0 wt %, from 20.0 wt % to 80.0 wt %, from 25.0 wt % to 75.0 wt %, from 30.0 wt % to 70.0 wt %, from 35.0 wt % to 65.0 wt %, from 40.0 wt % to 60.0 wt %, or from 45.0 wt % to 55.0 wt %. In some embodiments, the metal oxide catalyst component includes from 50.0 wt % to 80.0 wt % of the formed hybrid catalyst, such as from 50.0 wt % to 75.0 wt %, from 50.0 wt % to 70.0 wt %, from 60.0 wt % to 80.0 wt %, from 60.0 wt % to 75.0 wt %, or from 60.0 wt % to 70.0 wt %.

The metal oxide catalyst component and the microporous catalyst component may be combined with the mass ratio of from 1:10 to 10:1, from 1:10 to 9:1, from 1:10 to 8:1, from 1:10 to 5:1, from 1:10 to 4:1, from 1:10 to 3:1, from 1:8 to 8:1, from 1:8 to 7:1, from 1:8 to 6:1, from 1:8 to 5:1, from 1:8 to 4:1, from 1:5 to 8:1, from 1:5 to 7:1, from 1:5 to 6:1, or from 1:5 to 5:1.

After the metal oxide catalyst component has been prepared and combined with a microporous catalyst component, the binder is added to produce a paste. The binder may be capable of holding the metal oxide catalyst component and the microporous catalyst component together. The paste may be extruded to produce the formed hybrid catalyst. The formed hybrid catalyst may be formed by any suitable extrusion process.

Various binders are considered suitable. For example, the binder may include alumina, zirconia, or both. In embodiments, the binder may include pure alumina. In embodiments, the binder may include pure zirconia. When the binder includes alumina, the alumina binder may be a hydrous alumina. A hydrous alumina composition may be prepared from bohemitic precursors with water and peptizing agent. The binder may be mixed with the metal oxide catalyst component and the microporous catalyst component. After mixing the binder with the metal oxide catalyst component and the microporous catalyst component, the mixture may be extruded, dried, and calcined. After calcination, the binder may form aluminum oxide and bind the metal oxide catalyst component and the microporous catalyst component together to provide mechanical strength to extrude the formed hybrid catalyst. Without being bound by any particular theory, other typically employed binders, such as SiO₂ and TiO₂, may lead to poisoning of the catalyst activity or significant loss in olefin selectivity. The combination of the two catalyst components into a single catalyst body is not trivial. While a physical mixture of the metal oxide catalyst component and the microporous catalyst component (i.e., not formed into a single catalyst body) may reduce the pressure drop over the reactors, the catalytic performance, such as olefin selectivity, and carbon conversion, drops dramatically.

The binder including alumina, zirconia, or both, can combine the metal oxide catalyst component and the microporous catalyst component into a single catalyst body to improve C₂ to C₄ olefin yields and carbon conversion. Individually forming both metal oxide catalyst and microporous catalyst and combining them as a physical mixture is not able to obtain C₂ to C₄ and carbon conversion that are obtained with a formed hybrid catalyst as disclosed and described herein.

In embodiments, the binder is a colloidal solution, suspension, or gel of a binder precursor. The binder precursor may include oxides or hydroxides of aluminium, oxides or hydroxides of zirconium, or mixtures thereof. In one embodiment, the binder precursor may include pure alumina, (pseudo) boehmite or gibbsite, or mixtures thereof. In other embodiments, the binder precursor may include pure zirconia, hydrous zirconia, or mixtures thereof.

In embodiments, when the binder includes alumina, the binder may have [H⁺]/[Al] ratio of from 0.005 to 0.1, from 0.01 to 0.1, or about 0.05.

In embodiments, the binder may have a surface area of from 100 m²/g to 400 m²/g, from 125 m²/g to 400 m²/g, from 150 m²/g to 400 m²/g, from 100 m²/g to 200 m²/g, from 125 m²/g to 200 m²/g, from 150 m²/g to 200 m²/g, from 100 m²/g to 175 m²/g, from 125 m²/g to 175 m²/g, from 150 m²/g to 175 m²/g, from 100 m²/g to 150 m²/g, from 125 m²/g to 150 m²/g, or from 100 m²/g to 125 m²/g.

Without being bound by any particular theory, the use of templated molecular sieves (e.g. uncalcined) for the formulation has been found to have a positive impact on the catalyst performance and structural properties, particularly when strongly acidic conditions, such as an [H⁺]/[Al] ratio of more than 0.05, or more than 0.025, are used during the formulation procedure of formed hybrid catalysts.

In some embodiments, mixed metal oxide catalyst component and/or the binder are substantially free of silica. The term “substantially free” of a constituent refers less than 0.5 weight percent (wt. %) of that component in a composition. For example, the mixed metal oxide catalyst component and binder that are substantially free of silica may have less than 0.5 wt. % silica based on the combined weight of the mixed metal oxide and binder.

The formed hybrid catalyst may be used in methods for converting carbon in a carbon-containing feed stream to C₂ to C₄ hydrocarbons. Such processes will be described in more detail below.

According to embodiments, a feed stream is fed into a reaction zone, the feed stream comprising hydrogen (H₂) gas and a carbon-containing gas selected from carbon monoxide (CO), carbon dioxide (CO₂), and combinations thereof. In some embodiments, the H₂ gas is present in the feed stream in an amount of from 10 volume percent (vol %) to 90 vol %, based on combined volumes of the H₂ gas and the gas selected from CO, CO₂, and combinations thereof. The feed stream is contacted with a formed hybrid catalyst as disclosed and described herein in the reaction zone. The formed hybrid catalyst includes a metal oxide catalyst component comprising gallium oxide and zirconia, a microporous catalyst component, and a binder.

It should be understood that the activity of the formed hybrid catalyst will be higher for feed streams containing CO as the carbon-containing gas, and that the activity of the formed hybrid catalyst decreases as a larger portion of the carbon-containing gas in the feed stream is CO₂. However, that is not to say that the formed hybrid catalyst disclosed and described herein cannot be used in methods where the feed stream includes CO₂ as all, or a large portion, of the carbon-containing gas.

The feed stream is contacted with the formed hybrid catalyst in the reaction zone under reaction conditions sufficient to form a product stream comprising C₂ to C₄ hydrocarbons. The reaction conditions include a temperature within the reaction zone ranging, according to one or more embodiments, from 350° C. to 480° C., from 375° C. to 450° C., from 400° C. to 450° C., from 350° C. to 425° C., from 375° C. to 425° C., from 400° C. to 425° C., from 350° C. to 400° C., or from 375° C. to 400° C.

In embodiments, the reaction conditions include a pressure inside the reaction zone of at least 1 bar (100 kilopascals (kPa), such as at least 5 bar (500 kPa), at least 10 bar (1,000 kPa), at least 15 bar (1,500 kPa), at least 20 bar (2,000 kPa), at least 25 bar (2,500 kPa), at least 30 bar (3,000 kPa), at least 35 bar (3,500 kPa), at least 40 bar (4,000 kPa), at least 45 bar (4,500 kPa), at least 50 bar (5,000 kPa), at least 55 bar (5,500 kPa), at least 60 bar (6,000 kPa), at least 65 bar (6,500 kPa), at least 70 bar (7,000 kPa), at least 75 bar (7,500 kPa), at least 80 bar (8,000 kPa), at least 85 bar (8,500 kPa), at least 90 bar (9,000 kPa), at least 95 bar (9,500 kPa), or at least 100 bar (10,000 kPa). In other embodiments, the reaction conditions include a pressure inside the reaction zone is from 5 bar (500 kPa) to 100 bar (10,000 kPa), such as from 10 bar (1,000 kPa) to 95 bar (9,500 kPa), from 15 bar (1,500 kPa) to 90 bar (9,000 kPa), from 20 bar (2,000 kPa) to 85 bar (8,500 kPa), from 25 bar (2,500 kPa) to 80 bar (8,000 kPa), from 30 bar (3,000 kPa) to 75 bar (7,500 kPa), from 35 bar (3,500 kPa) to 70 bar (7,000 kPa), from 40 bar (4,000 kPa) to 65 bar (6,500 kPa), from 45 bar (4,500 kPa) to 60 bar (6,000 kPa), or from 50 bar (5,000 kPa) to 55 bar (5,500 kPa). In some embodiments, the pressure inside the reaction zone is from 20 bar (2,000 kPa) to 60 bar (6,000 kPa).

According to embodiments, the gas hourly space velocity (GHSV) within the reaction zone is from 500 per hour (/h) to 12,000/h, such as from 500/h to 10,000/h, from 1,200/h to 12,000/h, from 1,500/h to 10,000/h, from 2,000/h to 9,500/h, from 2,500/h to 9,000/h, from 3,000/h to 8,500/h, from 3,500/h to 8,000/h, from 4,000/h to 7,500/h, from 4,500/h to 7,000/h, from 5,000/h to 6,500/h, or from 5,500/h to 6,000/h. In some embodiments the GHSV within the reaction zone is from 1,800/h to 3,600/h, such as from 2,000/h to 3,600/h, from 2,200/h to 3,600/h, from 2,400/h to 3,600/h, from 2,600/h to 3,600/h, from 2,800/h to 3,600/h, from 3,000/h to 3,600/h, from 3,200/h to 3,600/h, or from 3,400/h to 3,600/h. In some embodiments, the GHSV within the reaction zone is from 1,800/h to 3,400/h, such as from 1,800/h to 3,200/h, from 1,800/h to 3,000/h, from 1,800/h to 2,800/h, from 1,800/h to 2,600/h, from 1,800/h to 2,400/h, from 1,800/h to 2,200/h, or from 1,800/h to 2,000/h. In some embodiments, the GHSV within the reaction is from 2,000/h to 3,400/h, such as from 2,200/h to 3,200/h, from 2,400/h to 3,000/h, or from 2,600/h to 2,800/h.

Comparing to individually formed catalytic functions, by using formed hybrid catalysts disclosed and described herein along with the process conditions disclosed and described herein, improved C₂ to C₄ olefin yields and carbon conversion may be achieved. For example, in embodiments where hydrogen to carbon monoxide H₂/CO ratios range from 2 to 5, such as greater than 2.2 and less than 3.8, or greater than 2.8 and less than 3.4, where temperatures range from 360° C. to 460° C., such as from 380° C. to 440° C., from 400° C. to 420° C., or from 400° C. to 410° C., pressure ranges from 5 to 100 bars, such as from 20 to 80 bars, or from 30 to 60 bars, and weight hourly space velocity ranges from 1 hr⁻¹ to 5 hr⁻¹, such as from 1 hr⁻¹ to 3 hr⁻¹. Using such conditions, the C₂ to C₄ olefin yield is greater than or equal to 4.0 mol %, such as greater than or equal to 5.0 mol %, greater than or equal to 7.0 mol %, greater than or equal to 10.0 mol %, greater than or equal to 12.0 mol %, greater than or equal to 15.0 mol %, greater than or equal to 17.0 mol %, greater than or equal to 20.0 mol %, greater than or equal to 22.0 mol %, greater than or equal to 25.0 mol %, greater than or equal to 27.0 mol %, greater than or equal to 30.0 mol %, greater than or equal to 32.0 mol %, greater than or equal to 35.0 mol %, greater than or equal to 37.0 mol %, greater than or equal to 40.0 mol %, greater than or equal to 42.0 mol %, greater than or equal to 45.0 mol %, greater than or equal to 47.0 mol %, greater than or equal to 50.0 mol %, greater than or equal to 52.0 mol %, greater than or equal to 55.0 mol %, greater than or equal to 57.0 mol %, greater than or equal to 60.0 mol %, greater than or equal to 62.0 mol %, greater than or equal to 65.0 mol %, greater than or equal to 67.0 mol %, greater than or equal to 70.0 mol %, greater than or equal to 72.0 mol %, greater than or equal to 75.0 mol %, greater than or equal to 77.0 mol %, greater than or equal to 80.0 mol %, greater than or equal to 82.0 mol %. In some embodiments, the maximum C₂ to C₄ olefin yield is 85.0 mol %. Thus, in some embodiments, the C₂ to C₄ olefin yield is from greater than or equal to 4.0 mol % to 85.0 mol %, such as from 5.0 mol % to 85.0 mol %, from 7.0 mol % to 85.0 mol %, from 10.0 mol % to 85.0 mol %, from 12.0 mol % to 85.0 mol %, from 15.0 mol % to 85.0 mol %, from 17.0 mol % to 85.0 mol %, from 20.0 mol % to 85.0 mol %, from 22.0 mol % to 85.0 mol %, from 25.0 mol % to 85.0 mol %, from 27.0 mol % to 85.0 mol %, from 30.0 mol % to 85.0 mol %, from 32.0 mol % to 85.0 mol %, from 35.0 mol % to 85.0 mol %, from 37.0 mol % to 85.0 mol %, or from 40.0 mol % to 85.0 mol %.

In embodiments, using formed hybrid catalysts disclosed and described herein along with the process conditions disclosed and described herein, the carbon conversion may be improved. Within the process ranges disclosed, the conversion of the feed containing carbon oxides and hydrogen can be carried out in a series of rectors with an intermediate knock-out of water by-product by the means of e.g., phase separation, membrane separation, or some type of water-selective absorptive or adsorptive process. Further directing the partially converted and water-free effluent to the subsequent reactor in series and repeating this manner of technological operations will have an overall effect of enhancing the olefin yield.

In embodiments, using formed hybrid catalysts disclosed and described herein along with the process conditions disclosed and described herein, the process may have C₂-C₃ olefin selectivity/paraffin selectivity ratio of greater than or equal to 2, from 2 to 20, from 2 to 10, from 2 to 8, from 2 to 6, from 3 to 11, from 3 to 10, from 3 to 8, from 3 to 6, or about 4.

In addition to improved selectivity, yield, and conversion at extended times on stream, using formed hybrid catalyst according to embodiments also provides these benefits across a wider range of process conditions (temperature, pressure, flow rate, etc.) in the reaction zone of a reactor. For example, the formed hybrid catalyst according to embodiments disclosed and described herein can allow lower reaction temperatures to be used while still providing high conversion, selectivity, yield, and low oxygenate selectivity over time on stream.

### EXAMPLES

The following examples illustrate features of the present disclosure but are not intended to limit the scope of the disclosure. For each of the following examples and comparative examples, the microporous catalyst component was prepared as follows: SAPO-34 was synthesized per literature procedures (Lok, B. M.; Messina, C. A.; Patton, R. L.; Gajek, R. T.; Cannan, T. R.; Flanigen, E. M. Crystalline silicoaluminophosphates. U.S. Pat. No. 4,440,871A, 1984). When using calcined SAPO-34, the materials was calcined in air using the following program: 25° C. raise to 600° C. at a heating rate of 5° C./min, hold at 600° C. for 4 hours (h), cool down to 25° C. in 4 h. The material was sieved to a fraction smaller than 200 mesh (smaller than 75 μm).

### Example 1

A metal oxide catalyst component comprising gallium on a zirconia support was prepared by an incipient wetness impregnation method. An impregnation solution of Gallium(III) nitrate hydrate (Ga(NO₃)₃·xH₂O) and Lanthanum(III) nitrate hexahydrate (La(NO₃)₃·6H₂O) with a concentration of respectively 1 mol/L and 0.3 mol/L in DI water was prepared. 5 g of smaller than 200 mesh size (smaller than 75 μm) ZrO₂ support (manufactured by NORPRO, product code SZ31164, BET surface area=100 m²/g, more than 95% monoclinic phase by XRD, pore volume=0.4 mL/g measured by DI water) was weighed and placed into a glass vial. After that, 2 mL of the Ga and La impregnation solution was added dropwise to the support while constantly shaking. After impregnation, the metal oxide catalyst component was dried at 85° C. in a forced convection oven overnight and calcined in a muffle furnace using the following program: from 25° C. to 550° C. at a heating rate of 3° C./min held at 550° C. for 4 hours. After calcination the catalyst was re-sieved to smaller than 200 mesh size (smaller than 75 μm) to remove larger agglomerated particles.

The powder was prepared by mixing 3.75 g of the metal oxide catalyst component described above with 1.25 g of calcined SAPO-34 (smaller than 200 mesh size, smaller than 75 μm) for 10 min using a mortar and pestle. Separately, pseudoboehmite (AlOOH) (manufactured by Sasol Limited, tradename Catapal D) was peptized in water using HNO₃ (65 wt. % in H₂O) at a HNO₃/Al ratio of 0.05, and a total solid content of 27 wt. %. The peptized pseudoboehmite mixture was added to the dried powders to form a paste, targeting a pseudoboehmite concentration of 20 wt. % on total solids basis (Catapal D, SAPO-34 and MMO). The paste was subsequently mixed for at least 10 minutes using the mortar and pestle until an extrudable paste was obtained. The paste was transferred to a ceramic dish and dried at 85° C. overnight to form a dried precursor. The dried precursor was heated from 25° C. to 600° C. at a heating rate of 2° C./min in a static muffle furnace and held at 600° C. for 4 hours to form a formed hybrid catalyst. After calcination, the formed hybrid catalyst was crushed and sieved to 40 mesh (400 μm) to 80 mesh (177 μm) for testing.

### Example 2

The formed hybrid catalyst was prepared according to Example 1, but the formed hybrid catalyst was re-sized from 20 mesh (841 μm) to 30 mesh (575 μm) for testing.

### Example 3

The metal oxide catalyst component was prepared according to Example 1. The formed hybrid catalyst was prepared according to Example 1, but 3.35 g of the metal oxide catalyst component and 1.25 g of calcined SAPO-34 were mixed to prepare the formed hybrid catalyst. The formed hybrid catalyst was re-sized from 40 mesh (400 μm) to 80 mesh (177 μm) for the testing.

### Example 4

The metal oxide catalyst component was prepared according to Example 1. The hybrid catalyst was prepared by mixing 5 g of the metal oxide catalyst component described above with 2.5 g of calcined SAPO-34 (smaller than 200 mesh size, smaller than 75 μm) for 10 min using a mortar and pestle. To this mixture, 5 mL of zirconia sol (manufactured by Daiichi Kigenso Kagaku-Kogyo Co., Ltd., tradename ZSL-10A; 10 wt. % ZrO₂ basis), which is colloidal ZrO₂ solution, was added to form a paste. The paste was subsequently mixed for at least 10 minutes using the mortar and pestle until a kneadable paste was obtained. The paste was transferred to a ceramic dish and dried at 85° C. overnight to form a dried precursor. The dried precursor was heated from 25° C. to 600° C. at a heating rate of 2° C./min in a static muffle furnace and held at 600° C. for 4 hours to form a formed hybrid catalyst. After calcination, the formed hybrid catalyst was crushed and sieved to 40 mesh (400 μm) to 80 mesh (177 μm) for testing.

### Example 5

The metal oxide catalyst component was prepared according to Example 1. The formed hybrid catalyst was prepared according to Example 1, but 8 g of the metal oxide catalyst component and 1.808 g of uncalcined SAPO-34 were mixed to prepare the formed hybrid catalyst. A targeting pseudoboehmite concentration was 24.6 wt. % on total solids basis. The formed hybrid catalyst was re-sized from 20 mesh (841 μm) to 30 mesh (575 μm) for testing.

### Comparative Example 1

The metal oxide catalyst component was prepared according to Example 1, but after calcination the catalyst was re-sieved to 40 mesh (400 μm) to 80 mesh (177 μm) size to remove fine particles.

The hybrid catalyst was prepared by combining 1 g of the metal oxide catalyst component described above with 0.33 g of pre-calcined SAPO-34 (40-80 mesh size) and shaken for 30 see until well mixed.

### Comparative Example 2

The hybrid catalyst was prepared according to Comparative Example 1, but each of the particle size of the metal oxide catalyst and SAPO-34 was 20 mesh (841 μm) to 30 mesh (575 μm).

### Comparative Example 3

The metal oxide catalyst component was prepared according to Comparative Example 1.

The hybrid catalyst was prepared by mixing 5 g of the metal oxide catalyst component described above with 2.5 g of calcined SAPO-34 (smaller than 200 mesh size, smaller than 75 μm) for 10 min using a mortar and pestle. To this mixture, 2.925 mL of 40 wt. % aqueous dispersion of colloidal titanium dioxide (manufactured by Evonik Industries, tradename Aerodisp® W-740X) was added, together with 2.075 mL of deionized H₂O (Target 18 wt. % of TiO₂ binder). The components were subsequently mixed for at least 10 minutes using a mortar and pestle until a kneadable paste was obtained. The paste was transferred to a ceramic dish and dried at 85° C. overnight to form a dried precursor. The dried precursor was heated from 25° C. to 600° C. at a heating rate of 2° C./min in a static muffle furnace and held at 600° C. for 4 hours to form a hybrid catalyst. After calcination, the hybrid catalyst was crushed and sieved to 40 mesh (400 μm) to 80 mesh (177 μm) for testing.

### Comparative Example 4

The metal oxide catalyst component was prepared according to Comparative Example 1.

The hybrid catalyst was prepared by mixing 3.2 g of the metal oxide catalyst component described above with 0.724 g of uncalcined SAPO-34 (smaller than 200 mesh size, smaller than 75 μm) for 10 min using a mortar and pestle. To this mixture, 1.7 mL of alkaline 40 wt. % aqueous dispersion of colloidal silica (manufactured by Grace, tradename Ludox® AS-40) was added, together with 0.93 mL of deionized H₂O (Target 18 wt. % of SiO₂ binder). The components were subsequently mixed for at least 10 minutes using a mortar and pestle until a kneadable paste was obtained. The paste was transferred to a ceramic dish and dried at 85° C. overnight to form a dried precursor. The dried precursor was heated from 25° C. to 600° C. at a heating rate of 2° C./min in a static muffle furnace and held at 600° C. for 4 hours to form a hybrid catalyst. After calcination, the hybrid catalyst was crushed and sieved to 40 mesh (400 μm) to 80 mesh (177 μm) for testing.

### Comparative Example 5

The metal oxide catalyst component was prepared according to Comparative Example 1.

The hybrid catalyst was prepared by combining 0.8 g of the metal oxide catalyst component described above with 0.2 g of pre-calcined SAPO-34 (40-80 mesh size) and shaken for 30 see until well mixed.

### Catalytic Performance Data

Testing of the hybrid catalysts was performed in a stainless steel fixed bed reactor system (7.7 mm internal diameter) under the following conditions in Table 1. The volume of balance gas for was 10%.

TABLE 1

| | Temperature | H₂/CO | Pressure | Weight Hour Space |
|---|---|---|---|---|
| | (° C.) | ratio | (Barg) | Velocity (hr⁻¹) |
| Condition 1 | 420 | 3 | 50 | 1.54 |
| Condition 2 | 400 | 3 | 40 | 1.54 |
| Condition 3 | 400 | 3 | 40 | 1.37 |
| Condition 4 | 420 | 3 | 40 | 1.87 |
| Condition 5 | 420 | 3.3 | 40 | 2.79 |
| Condition 6 | 400 | 3.3 | 40 | 2.79 |
| Condition 7 | 430 | 2 | 40 | 3 |

In Conditions 1-4, the WHSV was kept constant on an equal active catalyst basis (WHSV_{(MMO+SAPO-34)}) whereas in Conditions 5-7 the total WHSV (e.g. including binder) was kept constant. Prior to contacting with syngas, the catalyst was heated under nitrogen (N₂) to reaction temperature and pressure. The reactor effluent composition was obtained by gas chromatography and the conversion and carbon based selectivities are calculated using the following equations:

WHSV_{(MMO+SAPO-34)}=(F_{CO}+F_{H2})/W_{MMO}+W_{SAPO-34}

WHSV=(F_{CO}+F_{H2})/W_{catalyst}

Where F_{CO} and F_{H2} are defined as the mass flow rates of CO and H₂ respectively, and W_{MMO}, W_{SAPO-34} and W_{catalyst} are defined as the mass of MMO component, mass of SAPO-34 component and total catalyst mass (including binder), respectively.

X_{CO} (%)=[(η_{CO,in}−η_{CO,out})/η_{CO,in}]·100; and (1)

Sⱼ (%)=[αⱼ·η_{j,out}/(η_{CO,in}−η_{CO,out})]·100, (2)
where X_{CO} is defined as the CO conversion (%), η_{CO, in} is defined as the molar inlet flow of CO (μmol/s), η_{CO, out} is the molar outlet flow of CO (μmol/s), Sⱼ is defined as the carbon based selectivity to product j (%), αⱼ the number of carbon atoms for product j, η_{j, out} is the molar outlet flow of product j (μmol/s). All data was collected under steady state conditions, after at least 40 hours time on stream.

The results of the catalytic testing are shown in Table 2 below.

TABLE 2

| | | | | | | C₂-C₃ | | |
|---|---|---|---|---|---|---|---|---|
| | | | | | | Olefin | | |
| | | Carbon | | C₂-C₅ | C₂-C₃ | Selectivity/ | | Methanol |
| | | Conversion | CH₄ | Hydrocarbon | Olefin | Paraffin | | Selectivity |
| | | (CO) (C | Selectivity | Selectivity | Selectivity | Selectivity | C₂/C₃ | (C mol |
| | Condition | mol %) | (C mol %) | (C mol %) | (C mol %) | ratio | ratio* | %) |
| Example 1 | 1 | 69.6 | 4.15 | 60.3 | 35.6 | 2.34 | 0.27 | 0.02 |
| Example 2 | 1 | 69.5 | 3.69 | 60.1 | 35.1 | 2.3 | 0.26 | 0.02 |
| Comparative | 1 | 73.7 | 4.32 | 56.1 | 32.5 | 1.66 | 0.23 | 0.03 |
| Example 1 | | | | | | | | |
| Comparative | 1 | 58.1 | 3.4 | 56.6 | 25.3 | 1.08 | 0.20 | 0.04 |
| Example 2 | | | | | | | | |
| Example 1 | 2 | 55.6 | 1.6 | 61.1 | 41.2 | 4.1 | 0.26 | <0.01 |
| Example 2 | 2 | 55.6 | 1.5 | 60.4 | 40.4 | 4.0 | 0.26 | <0.01 |
| Comparative | 2 | 57.1 | 2 | 51.5 | 35.8 | 3 | 0.27 | 0.04 |
| Example 1 | | | | | | | | |
| Comparative | 2 | 28.9 | 2.5 | 48.3 | 25 | 1.4 | 0.4 | 0.37 |
| Example 2 | | | | | | | | |
| Example 3 | 3 | 56.9 | 1.4 | 59.8 | 40.2 | 3.4 | 0.24 | <0.01 |
| Example 4 | 3 | 58.1 | 1.4 | 61.9 | 40.5 | 3.3 | 0.23 | 0.03 |
| Comparative | 3 | 44.0 | 2.8 | 59.1 | 25.2 | 1.1 | 0.22 | <0.01 |
| Example 3 | | | | | | | | |
| Comparative | 4 | 14.0 | 4.4 | 61.7 | 31.4 | 1.92 | 0.36 | <0.01 |
| Example 4 | | | | | | | | |
| Example 5 | 5 | 45.2 | 1.14 | 64.2 | 47.1 | 7.3 | 0.4 | 0.05 |
| | 6 | 33.5 | 1.2 | 63.2 | 46.7 | 10.1 | 0.4 | <0.01 |
| | 7 | 50.0 | 1.14 | 57.0 | 43.0 | 7.85 | 0.47 | 0.07 |
| Comparative | 5 | 45.4 | 1.24 | 61.7 | 40.6 | 3.63 | 0.34 | 0.07 |
| Example 5 | 6 | 26.5 | 1.4 | 51.9 | 33.4 | 3.25 | 0.39 | 1.3 |
| | 7 | 45.4 | 1.26 | 54.8 | 36.4 | 3.42 | 0.37 | 0.07 |

*C₂/C₃ ratio means total C₂ Hydrocarbons (Sum of Ethylene and Ethane Cmol selectivity)/total C₃ hydrocarbons (Sum of propylene and propane Cmol selectivity)

As shown in Table 2, the comparison of Examples 1-2 versus Comparative Examples 1-2 demonstrates the improved performance of the single pellet formulation compared to dual pellet mixtures when loading larger particles into the reactor. Whereas dual pellet mixtures suffer from transport limitations, the single pellet formulation retains identical performance between large and small particles loaded in the reactor. Additionally, higher olefin selectivities and C₂/C₃ ratios are obtained for the single pellet formulation while maintaining low oxygenate selectivity, leading to higher absolute ethylene yields. Single pellet formulations also show a wider window of operability, particularly lowering the operating temperature. As shown in Comparative Example 2 with Condition 2, dual pellet formulations suffer from high oxygenate selectivities when operated at 400° C.

The comparison of Examples 3-4 versus Comparative Examples 3-4 shows the importance of the used binder. Al₂O₃ and ZrO₂ binder give comparable performance, whereas TiO₂ and SiO₂ binder respectively lead to olefin hydrogenation or poisoning of the catalyst.

Example 5 shows the performance of a 4:1 single pellet formulation compared to a 4:1 dual pellet formulation in Comparative Example 5 under different reaction conditions. At equal total WHSV, the single pellet shows comparable conversion to the dual pellet system, but with significantly higher hydrocarbon and olefin selectivities as well as higher C₂/C₃ ratios. Additionally, the single pellet allows for reaction at lower temperature without oxygenate selectivity, as demonstrated in condition 6.

It is noted that one or more of the following claims utilize the term “where” or “in which” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.” For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used in the Specification and appended Claims, the singular forms “a”, “an”, and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

It should be understood that any two quantitative values assigned to a property may constitute a range of that property, and all combinations of ranges formed from all stated quantitative values of a given property are contemplated in this disclosure. The subject matter of the present disclosure has been described in detail and by reference to specific embodiments. It should be understood that any detailed description of a component or feature of one or more embodiments does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

1. A process for preparing C₂ to C₄ hydrocarbons comprising:
introducing a feed stream comprising hydrogen gas and a carbon-containing gas selected from the group consisting of carbon monoxide, carbon dioxide, and mixtures thereof into a reaction zone of a reactor; and
converting the feed stream into a product stream comprising C₂ to C₄ hydrocarbons in the reaction zone in the presence of a formed hybrid catalyst, the formed hybrid catalyst comprising:
a metal oxide catalyst component comprising gallium oxide and zirconia;
a microporous catalyst component that is a molecular sieve having 8-MR (Membered Ring) pore openings; and
a binder comprising alumina, zirconia, or both.

2. A process for preparing a formed hybrid catalyst comprising:
mixing a metal oxide catalyst component and a microporous catalyst component, wherein
the metal oxide catalyst component comprises gallium oxide and zirconia; and
the microporous catalyst component comprises a molecular sieve having 8-MR (Member Ring) pore openings;
adding a binder to the mixture of the metal oxide catalyst component and the microporous catalyst component to form a paste, wherein the binder is a colloidal solution, suspension, or gel of a binder precursor comprising oxides or hydroxides of aluminum, oxides or hydroxides of zirconium, or mixtures thereof; and
extruding the paste to produce the formed hybrid catalyst.

3. The process of claim 1, wherein the metal oxide catalyst component has a particle size of less than 150 μm.

4. The process of claim 1, wherein the metal oxide catalyst component further comprises lanthanum.

5. The process of claim 1, wherein the formed hybrid catalyst has a particle size of from 0.5 mm to 6 mm.

6. The process of claim 1, wherein the formed hybrid catalyst has a particle size from less than 1.5 mm to 3.0 mm.

7. The process of claim 1, wherein the metal oxide catalyst component comprises from 0.1 gallium oxide per 100 grams (g) zirconia to 30.0 g gallium oxide per 100 g of zirconia.

8. The process of claim 1, wherein the microporous catalyst component comprises SAPO-34.

9. The process of claim 1, wherein the microporous catalyst component comprises uncalcined SAPO-34.

10. The process of claim 1, wherein the binder comprises pure alumina.

11. The process of claim 1, wherein the binder comprises pure zirconia.

12. The process of claim 1, wherein the metal oxide catalyst component comprises from 40.0 weight ratio (wt. %) to 80.0 wt. % of the formed hybrid catalyst.

13. The process of claim 1, wherein the metal oxide catalyst component is formed by an impregnation method.

14. The process of claim 1, wherein a temperature within the reaction zone during the converting is from 350° C. (Celsius) to 480° C.

15. The process of claim 1, wherein the process has C₂-C₃ olefin selectivity/paraffin selectivity ratio of from 2 to 20.

16. The process of claim 1, the metal oxide catalyst component, the binder, or both the metal oxide component and the binder is substantially free of silica.
